(19) United States
(12) Patent Application Publication
(10) Pub. No.: US 2026/0257076 A1
(43) Pub. Date: Sep. 3, 2026

(54) FAR-INFRARED IRRADIATION DEVICE FOR MIDDLE EAR AND INNER EAR AND FAR-INFRARED IRRADIATION EARMUFF

(71) Applicant: Tsung-Ter KUO, HSINCHU (TW)

(72) Inventors: TSUNG-TER KUO, HSINCHU (TW); Huichung CHE, HSINCHU (TW); Tai-Hua HSIEH, Wuhan (HB)

(21) Appl. No.: 18/875,248

(22) PCT Filed: Aug. 29, 2024
(86) PCT No.: PCT/CN2024/115385

§ 371 Date: Dec. 16, 2024

(30) Foreign Application Priority Data
Oct. 30, 2023 (CN) 202311417038.4

(51) Int. Cl. A61N 5/06

(52) U.S. Cl. CPC A61N 5/0625; A61N 2005/063; A61N 2005/0647; A61N 2005/066

(57) ABSTRACT

The invention relates to the technical field of medical auxiliary instruments, in particular to a far-infrared irradiation device for a middle ear and an inner ear, which includes a conical element, a heating component and a waveguide; a large opening and a small opening are arranged in two axial ends of the conical element respectively, a conical surface is formed between the large opening and the small opening, and the conical element is connected to the waveguide through the small opening. Far-infrared rays emitted by a first far-infrared ray emitter can be diffused into the ear hole along the waveguide so as to promote resonance in the ear hole and promote blood circulation in the ear hole. Far-infrared rays emitted by a second far-infrared ray emitter can generate resonance to the position around the ear hole to promote blood circulation near the ear. Far-infrared rays can cover internal and external positions of the whole ear so that the coverage range is wider, and the effect for promoting blood circulation is better.

This application claims the priority of the Chinese patent application with the application No. 202311417038.4 filed on Oct. 30, 2023 and entitled “FAR-INFRARED IRRADIATION DEVICE FOR MIDDLE EAR AND INNER EAR AND FAR-INFRARED IRRADIATION EARMUFF”, the content of which is incorporated by reference herein.

### FIELD OF THE INVENTION

The invention relates to the technical field of medical auxiliary instruments, in particular to a far-infrared irradiation device for a middle ear and an inner ear and a far-infrared irradiation earmuff.

### BACKGROUND OF THE INVENTION

Otolaryngologists indicate that hearing generally begins to decline after the age of 50. Due to aging, metabolism decreases, and blood vessels in the inner ear become hardened or narrowed, which affects blood circulation. This hinders the delivery of nutrients to cells in the ear, affects microcirculation of the auditory nerve, and then causes hearing aging, or hardening of the auditory bones and deterioration of high-frequency sound reception. 70-80% of tinnitus is also caused by high-frequency hearing deterioration. To delay hearing aging, ears should be protected more in the daily life to prevent them from being damaged. In addition to nutritional supplements from food, promoting blood circulation is helpful for metabolism and improves blood circulation, having the effect of slowing down and improving hearing deterioration.

Far-infrared rays in the wavelength range of 8 μm to 12 μm can resonate with the molecules in a human body, promoting the expansion of capillaries, smoothing blood circulation, promoting metabolism, and thereby increasing the body's immunity. Therefore, in addition to applications in science and technology and astronomy, far-infrared rays are also used in medical and health care. For example, the irradiation of far-infrared rays is often used when blood stasis happens, or blood circulation is poor.

At present, in order to introduce far-infrared rays into small cavity structures of the human body, a far-infrared irradiation device generally guides the far-infrared rays optically and then converges the far-infrared rays into the ear through a waveguide. However, due to the small area of an ear hole, the area where the emitted far-infrared rays converged through the waveguide act will be limited, and thus the irradiation effect is poor; and the use of a large-area plane irradiator is not applicable due to the irregular structure and the small area of the ear.

### SUMMARY OF THE INVENTION

For the technical problems existing in the prior art, the present invention provides a far-infrared irradiation device for a middle ear and an inner ear, and a far-infrared irradiation earmuff to solve the problem that the irradiation effect of the prior far-infrared irradiation device irradiating far-infrared rays locally to the ear hole area is poor as mentioned above.

The technical scheme of the present invention for solving the technical problems mentioned above is: a far-infrared irradiation device for a middle ear and an inner ear, including: a conical element, a heating component and a waveguide, wherein a large opening and a small opening are respectively arranged at two axial ends of the conical element, a conical surface is formed between the large opening and the small opening, and the conical element is connected to the waveguide through the small opening, the large opening of the conical element is provided with a first far-infrared ray emitter for emitting far-infrared rays that converge from the large opening toward the small opening, and the conical surface is provided with a second far-infrared ray emitter for emitting far-infrared rays that diffuse outward from the conical surface;

the first far-infrared ray emitter includes a metal sheet and a first far-infrared radiation film, wherein the metal sheet is in contact with the conical element, and the first far-infrared radiation film is arranged on one side of the metal sheet close to the large opening;

the heating component is arranged on the other side of the metal sheet and is used to heat the metal sheet, so that the first far-infrared radiation film is heated to emit far-infrared rays; and the second far-infrared ray emitter includes a second far-infrared radiation film arranged on an outer surface of the conical surface of the conical element, wherein the second far-infrared radiation film is heated by the heat transmitted from the metal sheet through the conical element to emit far-infrared rays.

On the basis of the technical scheme above, the present invention has improvements below.

Further, a wavelength of either of the far-infrared rays emitted by either of the first far-infrared radiation film and the second far-infrared radiation film is 8 μm to 12 μm; and a temperature for heating the metal sheet is maintained at 39 to 45 degrees Celsius.

Further, the conical element is made of metal material, and the second far-infrared radiation film is coated on the outer surface of the conical surface.

Further, the conical element further includes a conical metal sheet coated on the outer surface of the conical surface, and the second far-infrared radiation film is coated on an outer surface of the conical metal sheet.

Further, an inner diameter of the large opening is D1, an inner diameter of the small opening is D2, a cone angle of the conical element is θ, and a shortest straight-line distance between the large opening and the small opening is D3, wherein D1, D2, θ and D3 satisfy:

$$\frac{D 1}{D 2} > 5 , D 3 = \frac{D 1 - D 2}{2 \times tan θ}$$

so that the far-infrared rays emitted by the first far-infrared ray emitter have an enhanced effect of

$${( \frac{D 1}{D 2} )}^{2}$$

on the waveguide; and the waveguide is in a shape of a hollow tube, with an inner diameter of D4, and D4 is not greater than D2.

Further, the cone angle θ is between 45 and 90 degrees.

Further, the cone angle θ is between 55 and 65 degrees.

Further, metal sheet is a flat sheet, and an inner surface of the conical element is further provided with a reflective film for reflecting the far-infrared rays emitted by the first far-infrared radiation film and converging theretoward the small opening.

Further, the metal sheet forms a spherical concave surface toward the large opening; and a radius of the spherical concave surface is D5, which satisfies a relation:

$$D 5 = \frac{D 3}{cos ( θ / 2 )} + \frac{D 2}{2 \cdot sin ( θ / 2 )} .$$

Further, the waveguide is made of a light-transmitting material.

Moreover, as compared with the prior art, the far-infrared irradiation device for the middle and inner ear provided by the present invention has at least the following beneficial effects: the far-infrared rays emitted by the first far-infrared ray emitter will irradiate into the ear hole along the waveguide to promote resonance inside the ear hole and thus promote blood circulation in the car hole; the far-infrared rays emitted by the second far-infrared ray emitter will generate resonance at the position around the ear hole and thus promote blood circulation around the ear; and the far-infrared rays can cover the entire inside and outside of the ear, with a wider coverage range and a better effect in promoting blood circulation.

The present invention further provides a far-infrared irradiation earmuff, including a pair of earmuff bodies, an elastic arm connecting the pair of earmuff bodies, and the far-infrared irradiation device for the middle and inner ear mentioned above, wherein each of the earmuff bodies is provided with the far-infrared irradiation device and a power input device; and the power input device is electrically connected to the heating component of the far-infrared irradiation device.

Moreover, as compared with the prior art, the far-infrared irradiation earmuff provided by the present invention has at least the following beneficial effects: the earmuff bodies can directly cover the human ears to ensure that the waveguide is aligned with the ear hole so that the far-infrared rays emitted by the first far-infrared ray emitter can accurately irradiate into the ear hole; at the same time, the far-infrared rays emitted to the position around the ear holes by the second far-infrared ray emitter; and the far-infrared rays can cover the entire ear area, and thus the irradiation effect is better, which has the functions of promoting ear blood circulation and activating cells, for example.

### BRIEF DESCRIPTION OF THE DRAWINGS

FIG. 1 is a schematic diagram of the overall structure of an embodiment of the present invention (wherein the metal sheet forms a spherical concave surface); and

FIG. 2 is a schematic diagram of the overall structure of another embodiment of the present invention (wherein the metal sheet is a flat sheet).

In the figures, the components represented by reference numerals are listed as follows:
1: a conical element;
1.1: a large opening;
1.2: a small opening;
1.3: a reflective film;
1.4: a conical surface;
1.41: a conical metal sheet;
2: a waveguide;
3: a first far-infrared ray emitter;
3.1: a metal sheet;
3.2: a first far-infrared radiation film;
3.3: a heating component;
4: a second far-infrared ray emitter; and
4.1: a second far-infrared radiation film.

### DETAILED DESCRIPTION OF THE PREFERRED EMBODIMENTS

The principles and features of the present invention are described below in conjunction with the figures. The examples given below are only used to explain the present invention but are not used to limit the scope of the present invention.

It should be noted that, unless otherwise clearly specified and defined, the proper nouns, “installed”, “connected” and “connecting”, should be understood in a broad sense. For example, it can be a fixed connection, a detachable connection, or an integrally molded structure. For one ordinarily skilled in the art, the specific meanings of such proper nouns in the present invention can be understood according to specific conditions.

Far-infrared rays, being a type of light wave, can be generally divided into near infrared rays in the wavelength range of 0.76 μm to 4 μm and far-infrared rays in the wavelength range of 4 μm to 400 μm. Clinical experiments have shown that biochemical far-infrared rays are similar to the human wavelength of 8 μm to 12 μm, and can easily resonate with the human body, having the functions of warming, massaging, promoting blood circulation, activating cells and so on, and are indispensable lights for animals and plants. Water accounts for 65-70% of the human body and about 60-95% of cells. The resonant active wavelength of water is about 8 μm to 10 μm.

In order to apply far-infrared rays to the human ear to promote metabolism and improve blood circulation, thereby slowing down and improving hearing deterioration, the present invention provides a far-infrared irradiation device for a middle ear and an inner ear, and a far-infrared irradiation earmuff to effectively protect the ear and delay hearing aging by applying far-infrared rays of corresponding wavelengths to the ears.

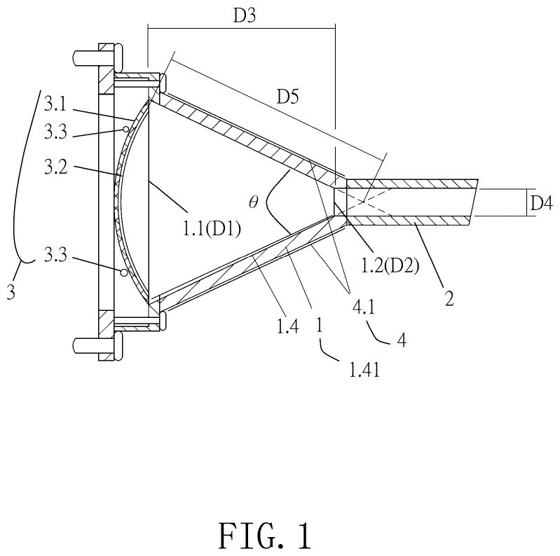

As shown in FIG. 1, the far-infrared irradiation device for the middle ear and inner ear designed by the present invention includes a conical element 1, a heating component 3.3, and a waveguide 2; a large opening 1.1 and a small opening 1.2 are respectively provided at the axial ends of the conical element 1, a conical surface 1.4 is formed between the large opening 1.1 and the small opening 1.2, and the conical element 1 is connected to the waveguide 2 through the small opening 1.2.

The large opening 1.1 of the conical element 1 is provided with a first far-infrared ray emitter 3 for emitting far-infrared rays that converge from the large opening 1.1 toward the small opening 1.2, and the conical surface 1.4 is provided with a second far-infrared ray emitter 4 for emitting far-infrared rays that diffuse outward from the conical surface 1.4.

The first far-infrared ray emitter 3 includes a metal sheet 3.1 and a first far-infrared radiation film 3.2, wherein the metal sheet 3.1 is in contact with the conical element 1, and the first far-infrared radiation film 3.2 is arranged on one side of the metal sheet 3.1 close to the large opening 1.1.

The heating component 3.3 is arranged on the other side of the metal sheet 3.1 and is used to heat the metal sheet 3.1 so that the first far-infrared radiation film 3.2 is heated to emit the far-infrared rays.

The second far-infrared ray emitter 4 includes a second far-infrared radiation film 4.1 arranged on an outer surface of the conical surface 1.4 of the conical element 1, wherein the second far-infrared radiation film 4.1 is heated by the heat transmitted from the metal sheet 3.1 through the conical element 1 to emit the far-infrared rays.

Specifically, the conical element 1 is a metal element. When the heating component 3.3 produces heat, part of the heat will directly act on the first far-infrared radiation film 3.2, and the remaining part of the heat will be conducted to the second far-infrared radiation film 4.1 through the conical element 1, thereby achieving the action of the two far-infrared radiation films at the same time.

Preferably, the conical element 1 is made of a metal material, and the second far-infrared radiation film 4.1 is coated on the outer surface of the conical surface 1.4 so that the thermal conductivity is better.

The far-infrared rays emitted by the first far-infrared ray emitter 3 will converge from the large opening 1.1 toward the small opening 1.2 of the conical element 1, and then be sent into the ear hole through the waveguide 2. The far-infrared rays will diverge into the ear hole along the waveguide 2 to promote resonance in the ear hole so as to promote blood circulation in the ear hole.

The far-infrared rays emitted by the second far-infrared ray emitter 4 diffuse outward from the conical surface 1.4 of the conical element 1 to produce resonance to the position around the ear hole, thereby promoting blood circulation around the ear.

In this embodiment, the far-infrared rays generated by the first far-infrared ray emitter 3 and the second far-infrared ray emitter 4 can cover the entire inside and outside of the ear so that the coverage range is wider and the effect of promoting blood circulation is better.

As an embodiment, the far-infrared rays emitted by either of the first far-infrared radiation film 3.2 and the second far-infrared radiation film 4.1 have a wavelength of 8 μm to 12 μm; the heating temperature of the metal sheet 3.1 is maintained at 39 to 45 degrees Celsius, preferably at 41±1 degrees Celsius.

Ideally, it is the better that the far-infrared ray emissivity is higher (100% emissivity), but it also needs to be in coordination with the wavelength and temperature of the object to be heated. Generally, the temperature of the human body is 37° C. According to Wien's displacement law, the most suitable wavelength is:

$$2897 ( a constant ) \div ( 273 + 37 ) ( an absolute temperature ) = 9.3 μm wavelenght .$$

Therefore, when far-infrared rays are used at 37° C., the higher the far-infrared ray release rate in the wavelength range of 9.3 μm is, the stronger the far-infrared ray power is and the better the effect is. In addition, the higher the temperature is, the stronger the power is.

The normal temperature of a human body is 36.5° C.~37° C., which is equivalent to a wavelength of about 9 μm to 9.5 μm. When an infrared product is heated to 41° C. to 50° C. and come into contact with the human body or skin, the far-infrared radiation will resonate with water molecules and bones of the human body.

At this time, the emissivity of the far-infrared rays at a wavelength of 9 μm to 9.5 μm will affect the degree of resonance of the far-infrared rays on the human body.

When the heating temperature is 41° C., the wavelength is:

$$2 \; 897 / ( 273 + 41 ) \approx 9.2 μm .$$

Unit power: Power=σT4, the higher the temperature is, the stronger the power is.

Therefore, considering the optimal wavelength and the operation temperature of a medical device, the heating temperature in this embodiment is set at 39~45° C., and optimally at 41+1° C.

In addition, Wien's displacement law is a law in physics that describes the inverse relationship between the peak wavelength of the radiance of a black body electromagnetic radiation spectrum and its own temperature, and the mathematical expression of which is:

$${λ}_{max} = \frac{b}{τ} ;$$

wherein λₘₐₓ is a peak wavelength of the radiation (unit: meter);
T is an absolute temperature of the black body (unit: Kelvin);
b is a proportionality constant, also known as a Wien displacement constant, and its value is equal to 2.8977729 (17)×10⁻³ m·k (the value recommended by the International Committee for Scientific and Technological Information in 2014, with an uncertainty digit in brackets at a confidence level of 68.27%).

In optics, nanometer (nm) is generally used as the unit of wavelength, so b=2.8977729 (17)×10⁻⁶ nm·k.

As a supplement, in the Stephen-Boltzmann law, it is stated that a total radiation power Eb of a black body is calculated as follows:

$${E}_{b} = {σ T}^{4} ;$$

wherein σ is a Stephen-Boltzmann constant and T is an absolute temperature of the black body.

The value of the Stephen-Boltzmann constant is 5.67×10⁻⁸ W/m²k⁴, or 3.3063×10⁻¹⁵ Btu/s·in²·F⁴.

The Planck distribution describes spectral variations of blackbody radiation. Integrating all of the wavelengths (2) with the Planck distribution law yields the Stephen-Boltzmann law.

When a black body with a surface area (A) is immersed in a medium with an ambient temperature of Ta, the calculation formula for the net rate of thermal radiation of the black body is as follows:

$${Q}_{radiation} = σ A ( {T}_{B}^{4} - {T}_{a}^{4} ) , {T}_{s} > {T}_{a} ;$$

wherein:
Tₛ=an absolute temperature of the black body;
Tₐ=an absolute temperature of a surrounding medium temperature (the ambient temperature).

As an embodiment, an inner diameter of the large opening 1.1 is D1, an inner diameter of the small opening 1.2 is D2, a cone angle of the conical element 1 is θ, and a shortest straight-line distance between the large opening 1.1 and the small opening 1.2 is D3, wherein the D1, the D2, the 0 and the D3 satisfy:

$$\frac{D 1}{D 2} > 5 , D 3 = \frac{D 1 - D 2}{2 \times tan θ} ,$$

so that the far-infrared rays emitted by the first far-infrared ray emitter 3 have an enhanced effect of

$${( \frac{D 1}{D 2} )}^{2}$$

on the waveguide 2; the waveguide 2 is in a shape of a hollow tube, with an inner diameter of D4, and D4 is not greater than D2, wherein the cone angle θ is between 45 and 90 degrees.

Preferably, the cone angle θ is between 55 and 65 degrees.

In this embodiment, the specifications of D1, D2, D3, D4 and the cone angle θ can be adjusted adaptively according to different user groups.

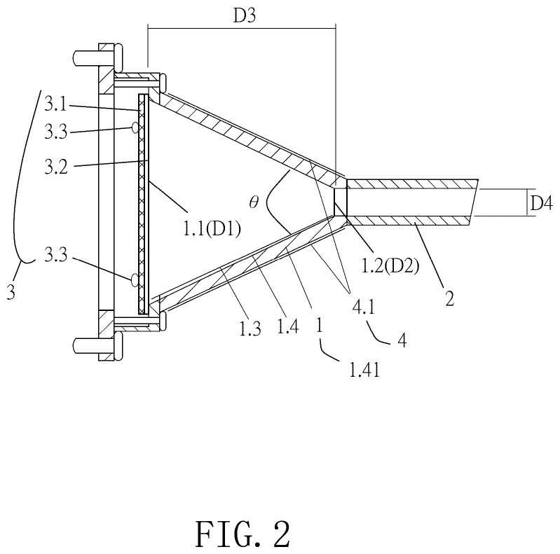

As an embodiment, specifically referring to FIG. 2, the metal sheet 3.1 is a flat sheet, and the inner surface of the conical element 1 is further coated with a reflective film 1.3 for reflecting the far-infrared rays emitted by the first far-infrared radiation film 3.2 and converging them toward the small opening 1.2 so as to ensure that the far-infrared rays emitted by the first far-infrared radiation film 3.2 can fully act in the ear hole.

As an embodiment, specifically referring to FIG. 1, the metal sheet 3.1 forms a spherical concave surface toward the large opening 1.1; and a radius of the spherical concave surface is D5, which satisfies a relation:

$$D 5 = \frac{D 3}{cos ( θ / 2 )} + \frac{D 2}{2 \cdot sin ( θ / 2 )} .$$

The center of the spherical concave surface falls on one side of the waveguide 2 close to the conical element 1, ensuring a better convergence effect of the far-infrared rays produced by the metal sheet 3.1.

As an embodiment, the waveguide 2 is made of a light-transmitting material to ensure that the far-infrared rays can be fully diffused to the ear hole.

The present invention further provides a far-infrared irradiation earmuff, including a pair of earmuff bodies, an elastic arm connecting the pair of earmuff bodies, and the far-infrared irradiation device for the middle and inner ear as described above; each of the earmuff bodies is provided with the far-infrared irradiation device and a power input device; and the power input device is electrically connected to the heating component 3.3 of the far-infrared irradiation device.

In this embodiment, the earmuff bodies can directly cover the human ears to ensure that the waveguide 2 is aligned with the ear holes so that the far-infrared rays emitted by the first far-infrared ray emitter 3 can accurately irradiate into the ear holes; at the same time, the far-infrared rays are emitted to the position around the ear holes by the second far-infrared ray emitter 4; and the far-infrared rays can cover the entire ear area to promote blood circulation in the ear, activate cells and so on.

It should be noted that, in the specification, the proper nouns “comprise”, “include” or any other variations thereof are intended to cover non-exclusive inclusion so that a process, method, article or apparatus that includes a series of elements includes not only those elements, but also other elements not explicitly listed, or also includes elements inherent to such a process, method, article or apparatus. Unless otherwise clearly specified and defined, the proper nouns, “installed”, “connected” and “connecting”, should be understood in a broad sense. For example, it can be a fixed connection, a detachable connection, or an integral connection; it can be a mechanical connection or an electrical connection; it can be a direct connection or an indirect connection through an intermediate medium; and it can be the internal connection of two elements. For one ordinarily skilled in the art, the specific meanings of such proper nouns in the present invention can be understood according to specific conditions.

Although preferred embodiments of the present invention are described, additional modifications and variations may be made to these embodiments once one ordinarily skilled in the art understands the basic inventive concepts. Therefore, it is intended that the appended claims be interpreted as including the preferred embodiments as well as all of the modifications and changes that fall within the scope of the present invention.

It is apparent that one skilled in the art can make various modifications and variations to the present invention without departing from the spirit and scope of the present invention. Thus, on the condition that these modifications and variations of the present invention belong to the scope of the claims in the present invention and their equivalents, the present invention intends to include these modifications and variations.

1. A far-infrared irradiation device for a middle ear and an inner ear, comprising a conical element (1), a heating component (3.3) and a waveguide (2), wherein a large opening (1.1) and a small opening (1.2) are respectively arranged at two axial ends of the conical element (1), a conical surface (1.4) is formed between the large opening (1.1) and the small opening (1.2), and the conical element (1) is connected to the waveguide (2) through the small opening (1.2), characterized in that:
the large opening (1.1) of the conical element (1) is provided with a first far-infrared ray emitter (3) for emitting far-infrared rays that converge from the large opening (1.1) toward the small opening (1.2), and the conical surface (1.4) is provided with a second far-infrared ray emitter (4) for emitting far-infrared rays that diffuse outward from the conical surface (1.4);
the first far-infrared ray emitter (3) comprises a metal sheet (3.1) and a first far-infrared radiation film (3.2), wherein the metal sheet (3.1) is in contact with the conical element (1), and the first far-infrared radiation film (3.2) is arranged on a side of the metal sheet (3.1) close to the large opening (1.1);
the heating component (3.3) is arranged on the other side of the metal sheet (3.1) and is used to heat the metal sheet (3.1) so that the first far-infrared radiation film (3.2) is heated to emit far-infrared rays; and
the second far-infrared ray emitter (4) comprises a second far-infrared radiation film (4.1) arranged on an outer surface of the conical surface (1.4) of the conical element (1), wherein the second far-infrared radiation film (4.1) is heated by the heat transmitted from the metal sheet (3.1) through the conical element (1) to emit far-infrared rays.

2. The far-infrared irradiation device for the middle ear and the inner ear according to claim 1, characterized in that a wavelength of the far-infrared rays emitted by either of the first far-infrared radiation film (3.2) and the second far-infrared radiation film (4.1) is 8 μm to 12 μm; and a temperature for heating the metal sheet (3.1) is maintained at 39 to 45 degrees Celsius.

3. The far-infrared irradiation device for the middle ear and the inner ear according to claim 2, characterized in that the conical element (1) is made of a metal material, and the second far-infrared radiation film (4.1) is coated on the outer surface of the conical surface (1.4).

4. The far-infrared irradiation device for the middle ear and the inner ear according to claim 2, characterized in that the conical element (1) further includes a conical metal sheet (1.41) coated on the outer surface of the conical surface (1.4), and the second far-infrared radiation film (4.1) is coated on an outer surface of the conical metal sheet (1.41).

5. The far-infrared irradiation device for the middle ear and the inner ear according to claim 2, characterized in that an inner diameter of the large opening (1.1) is D1, an inner diameter of the small opening (1.2) is D2, a cone angle of the conical element (1) is θ, and a shortest straight-line distance between the large opening (1.1) and the small opening (1.2) is D3,
wherein the D1, the D2, the θ and the D3 satisfy:
$$\frac{D 1}{D 2} > 5 , D 3 = \frac{D 1 - D 2}{2 \times tan θ} ,$$
so that the far-infrared rays emitted by the first far-infrared ray emitter (3) have an enhanced effect of
$${( \frac{D 1}{D 2} )}^{2}$$
on the waveguide (2); and
the waveguide (2) is in a shape of a hollow tube, with an inner diameter of D4, and D4 is not greater than D2.

6. The far-infrared irradiation device for the middle ear and the inner ear according to claim 5, characterized in that the cone angle θ is between 45 and 90 degrees.

7. The far-infrared irradiation device for the middle and the inner ear according to claim 6, characterized in that the cone angle θ is between 55 and 65 degrees.

8. The far-infrared irradiation device for the middle ear and the inner ear according to claim 5, characterized in that the metal sheet (3.1) is a flat sheet, and an inner surface of the conical element (1) is provided with a reflective film (1.3) for reflecting the far-infrared rays emitted by the first far-infrared radiation film (3.2) and converging theretoward the small opening (1.2).

9. The far-infrared irradiation device for the middle and the inner ear according to claim 5, characterized in that the metal sheet (3.1) forms a spherical concave surface toward the large opening (1.1); and a radius of the spherical concave surface is D5, which satisfies a relation:
$$D 5 = \frac{D 3}{cos ( θ / 2 )} + \frac{D 2}{2 \cdot sin ( θ / 2 )} .$$

10. The far-infrared irradiation device for the middle ear and an inner ear according to claim 4, characterized in that the waveguide (2) is made of a light-transmitting material.

11. The far-infrared irradiation earmuff, characterized by comprising a pair of earmuff bodies, an elastic arm connecting the pair of earmuff bodies, and the far-infrared irradiation device for the middle and inner ear according to claim 1; each of the earmuff bodies is provided with a power input device and the far-infrared irradiation device; and the power input device is electrically connected to the heating component (3.3) of the far-infrared irradiation device.

12. The far-infrared irradiation earmuff, characterized by comprising a pair of earmuff bodies, an elastic arm connecting the pair of earmuff bodies, and the far-infrared irradiation device for the middle and inner ear according to claim 2; each of the earmuff bodies is provided with a power input device and the far-infrared irradiation device; and the power input device is electrically connected to the heating component (3.3) of the far-infrared irradiation device.

13. The far-infrared irradiation earmuff, characterized by comprising a pair of earmuff bodies, an elastic arm connecting the pair of earmuff bodies, and the far-infrared irradiation device for the middle and inner ear according to claim 3; each of the earmuff bodies is provided with a power input device and the far-infrared irradiation device; and the power input device is electrically connected to the heating component (3.3) of the far-infrared irradiation device.

14. The far-infrared irradiation earmuff, characterized by comprising a pair of earmuff bodies, an elastic arm connecting the pair of earmuff bodies, and the far-infrared irradiation device for the middle and inner ear according to claim 4; each of the earmuff bodies is provided with a power input device and the far-infrared irradiation device; and the power input device is electrically connected to the heating component (3.3) of the far-infrared irradiation device.

15. The far-infrared irradiation earmuff, characterized by comprising a pair of earmuff bodies, an elastic arm connecting the pair of earmuff bodies, and the far-infrared irradiation device for the middle and inner ear according to claim 5; each of the earmuff bodies is provided with a power input device and the far-infrared irradiation device; and the power input device is electrically connected to the heating component (3.3) of the far-infrared irradiation device.

16. The far-infrared irradiation earmuff, characterized by comprising a pair of earmuff bodies, an elastic arm connecting the pair of earmuff bodies, and the far-infrared irradiation device for the middle and inner ear according to claim 6; each of the earmuff bodies is provided with a power input device and the far-infrared irradiation device; and the power input device is electrically connected to the heating component (3.3) of the far-infrared irradiation device.

17. The far-infrared irradiation earmuff, characterized by comprising a pair of earmuff bodies, an elastic arm connecting the pair of earmuff bodies, and the far-infrared irradiation device for the middle and inner ear according to claim 7; each of the earmuff bodies is provided with a power input device and the far-infrared irradiation device; and the power input device is electrically connected to the heating component (3.3) of the far-infrared irradiation device.

18. The far-infrared irradiation earmuff, characterized by comprising a pair of earmuff bodies, an elastic arm connecting the pair of earmuff bodies, and the far-infrared irradiation device for the middle and inner ear according to claim 8; each of the earmuff bodies is provided with a power input device and the far-infrared irradiation device; and the power input device is electrically connected to the heating component (3.3) of the far-infrared irradiation device.

19. The far-infrared irradiation earmuff, characterized by comprising a pair of earmuff bodies, an elastic arm connecting the pair of earmuff bodies, and the far-infrared irradiation device for the middle and inner ear according to claim 9; each of the earmuff bodies is provided with a power input device and the far-infrared irradiation device; and the power input device is electrically connected to the heating component (3.3) of the far-infrared irradiation device.

20. The far-infrared irradiation earmuff, characterized by comprising a pair of earmuff bodies, an elastic arm connecting the pair of earmuff bodies, and the far-infrared irradiation device for the middle and inner ear according to claim 10; each of the earmuff bodies is provided with a power input device and the far-infrared irradiation device; and the power input device is electrically connected to the heating component (3.3) of the far-infrared irradiation device.
